(19) United States
(12) Patent Application Publication
(10) Pub. No.: US 2014/0075604 A1
(43) Pub. Date: Mar. 13, 2014

(54) METHOD FOR MAKING A TURF OF STOLONIFEROUS OR RHIZOMATOUS PLANT SPECIES

(71) Applicants: Marco Volterrani, San Giuliano Terme-Pi (IT); Maurizio Pacini, San Giuliano Terme-Pi (IT)

(72) Inventors: Marco Volterrani, San Giuliano Terme-Pi (IT); Maurizio Pacini, San Giuliano Terme-Pi (IT)

(21) Appl. No.: 14/111,184

(22) PCT Filed: Apr. 7, 2011
(86) PCT No.: PCT/IB11/51508

§ 371 Date: Nov. 26, 2013

(30) Foreign Application Priority Data
Apr. 7, 2010 (IT) PI2010A000043

(51) Int. Cl. A01G 1/00; A01H 5/12

(52) U.S. Cl. CPC A01G 1/002; A01H 5/12
USPC 800/298; 435/430; 504/182; 504/181; 504/177; 504/183; 47/58.1 R

(57) ABSTRACT

Method for per to provide a turf for sports, ornamental or recreational use, comprising the steps of: selecting a stoloniferous and/or rhizomatous plant species; cultivating a plurality of mother plants belonging to said stoloniferous and/or rhizomatous species up to reach a predetermined vegetative development, said mother plants comprising stolons and/or rhizomes having a plurality of nodes, wherein two successive nodes are connected by plant portions, or internodes; preparing at least one cutting from each mother plant of said plurality of mother plants, said cutting comprising at least one node and an internode, wherein said internode has a length set between 1 mm and 10 mm; and carrying out a vegetative propagation of a plurality of said cuttings in a desired site then obtaining said turf.

### FIELD OF THE INVENTION

The present invention relates to a method for making a turf for sporting, recreational or ornamental purposes.

For example, a turf, according to the invention, can be used as a surface on which to play sports like golf, soccer, tennis, hockey, football, athletics, rugby, baseball, and other sports that require turf fields in general, as well as for ornamental use.

### BACKGROUND OF THE INVENTION

The known systems for making turfs provide can be selected among seeding, transplantation of parts of turfs, pre-rooted seedlings, stolons or installation of turf in rolls known as “at ready effect”.

It is also known that there are numerous varieties of macrotherm species for making turf that cannot be propagated by seeding because they are sterile hybrids, or because they produce seeds in a very low amount.

In case of use of plant parts for making turfs, instead, both preservation and transport are difficult. Therefore, it is necessary to use refrigerating means. Furthermore, once the parts of plant are distributed on the site for achieving carrying out a vegetative propagation, they require care and frequent irrigations.

A further technique provides installation of little clods. However, in this case, a long time is necessary to complete the turf. Furthermore, the final turf is usually not planar for the presence of reliefs caused by the original little clods.

The system of pre-rooted seedlings, even though it ensures a good rooting and an easy management, instead, requires a lot of manual work for preparing the seedlings.

The use of rolled turf has the advantage to provide a ready effect, but it is expensive and causes the removal of soil from the place of cultivation, and it cannot be used in case of firm ground.

In WO2006/067557 a method is described for making a turf. The method provides a seedling tray with a plurality of cells of growth. Each cell of growth has a hole made on the basis adapted to allow the outflow of irrigation water during the step of cultivating the plant species. In particular, the method provides introduction in each cell a predetermined amount of growth substrate containing the nutritive substances necessary to ensure the development of a seedling of herbaceous species ready for being transplanted in a chosen implant site, once achieved a status of appropriate ripening. The seedling can be obtained starting from seeds, or alternatively, starting from plant parts.

A method is also known, see for example “Nutrient alginate encapsulation of in vitro nodal segments of pomegranate for germplasm distribution and exchange” in the name of NAIK S K et al, for exploiting the beneficial effects of encapsulating axenic nodal segments of pomegranate in calcium alginate, or other, for exchanging and distributing germplasm. More in detail, in the article a process is described for encapsulating into a hydrogel of calcium alginate nodal segments obtained by cultivation “in vitro” of buds derived from explants of adult nodes, or from axenic cotyledon nodes of pomegranate, or other arboreal fruit species. The encapsulated nodes are described as able to germinate only if kept for about 20 days at a temperature of about 4° C. in specific culture substrates.

In case of hybrid grass, i.e. which cannot be reproduced by seeding, a procedure exists that provides distributing plant fragments directly on the cultivation field. The fragments, or portions, of the plant comprise indiscriminately nodes and internodes and are periodically collected and redistributed on the same cultivation field for regenerating it.

However, this procedure produces high amount of plant portions that are not able to germinate, in particular those comprising exclusively internodes. These parts of plants, therefore, tend quickly to wither and dry and their decomposition jeopardizes the survival of the those plant portions having nodes and that could, instead, germinate.

### SUMMARY OF THE INVENTION

It is therefore a feature of the present invention to provide a method for making a turf that is adapted to overcome the difficulties of the known techniques.

It is another feature of the present invention to provide a method for making a turf with species that can be propagated only in a vegetative way, which assists the operations of transporting, preserving as well as assisting the bedding steps in the implantation site.

It is a further feature of the present invention to provide loose products for carrying out a vegetative propagation of species adapted to make a turf, in particular of plant species that cannot be propagated by seeding, such as the hybrid lawn grass, the species belonging to the type Zoysia, Stenotaphrum and similar plant species.

These and other features are accomplished with one exemplary method for making a turf for sports, ornamental or recreational use comprising the steps of:
selecting a stoloniferous and/or rhizomatous plant species;
cultivating a plurality of mother plants belonging to said stoloniferous and/or rhizomatous species up to reach a predetermined vegetative development, said mother plants comprising stolons and/or rhizomes having a plurality of nodes, wherein two successive nodes are connected by internode portions, or internodes;
obtaining at least one cut portion from each mother plant of said plurality of mother plants, said cut portion comprising at least one node and one internode, wherein said internode has a length set between 1 mm and 10 mm;
carrying out a vegetative propagation of a plurality of said cuttings in a desired site then obtaining said turf.

In particular, according to the invention, the stolons of the mother plants are cut creating a plurality of cuttings each of witch comprising at least one node and at least one internode with length set between 1 mm and 10 mm, and all the cuttings are gathered creating a loose product, also definable as “pseudo-seed”, since comparable for size to a seed. The pseudo-seeds can be preserved for being used with processes of seeding, in particular by means of machines for seeding.

More in detail, the reduced size of the cutting, comprising at least one node and one internode with length set between 1 mm and 10 mm is comparable to the size of a seed, and allows then the use of seeding machines, which are capable of seeding a single seed at a time directly in the ground, in seedling containers, etc.

In fact, the node contains the meristems, or first-nodes, for generating roots and buds and then a new plant. More in detail, the nodes are clones of the plant which has generated them but are comparable to a seed concerning size, regenerative capacity, preservation mode, with the difference that a real seed would generate an individual that is genetically different from the individuals that have generated it. Instead, the node can be considered a “pseudo-seed” genetically equal to the plant that has generated it.

The presence of the internode set between 1 and 10 mm, in addition to be very reduced and then capable of giving to the cutting a shape and a size comparable to that of a seed is relevant, since the internode that is closest to the node contains nutritive substances, in particular amides and sugars, necessary and useful to the node for germinating. Instead, when the cutting consists only of a node, it is not able to germinate, or it germinates very slowly. On the other hand, if the cutting comprises one, or more internodes of length larger than 10 mm, it is much more easily attackable by fungi, parasites, etc. Therefore, a cutting that comprises a node, or a node portion, and an internode of length set between 1 mm and mm, is surprisingly capable of germinating and of rooting, and is less exposed to attacks of fungi, insects and parasites.

Preferably, the selected plant species belongs to the family of the hybrid grasses, i.e. of the grasses not capable of reproducing by seed. In particular, with carrying out a vegetative propagation the transplantation is intended of the cuttings, or pseudo-seeding, because the cuttings obtained as described above, are pseudo-seeds since they are comparable for size to a seed.

In particular, the cultivating step of the mother plant can be carried out in a greenhouse, or in open field.

Alternatively, the cultivating step of the mother plant is carried out in a container.

For example the container can be a seedling container comprising a plurality of cells, said cultivating step comprising in this case:
a step of putting a cutting, or pseudo-seed, of said stoloniferous and/or rhizomatous plant species in said cells of said seedling container;
an extraction step of said mother plants from said cell of said seedling container once achieved said predetermined vegetative growth.

Preferably, the internode has a length set between 1 mm and 5 mm.

In particular, the cuttings obtained as loose product are introduced into a container for their preservation and/or their transportation and/or sale.

In particular, once obtained the cuttings, or pseudo-seeds, they are kept at a temperature set between −5° C. and 8° C. before starting the step of carrying out a vegetative propagation on the implantation site.

In particular, before the seeding step, the mother plant can be treated with a vegetative growth regulator, in particular a growth retardant, selected from the group consisting of: Trinexapac-ethyle, Calcium-proesadione, Paclobutrazol, Uniconazole, Miconazole, Mepiquat Chloride, Chlorocholine Chloride, or Clormequat, Flurprimidol, Mefluidide, or a combination thereof. This way, stolons or rhizomes are obtained with nodes very close to each other and separated from very short internodes, facilitating, in particular, the automatic cutting process of the cuttings from the mother plants.

Advantageously, the step of obtaining of the cutting is made through a cutting step of stolons and/or rhizomes of the mother plant.

In particular, the cutting step provides a plurality of equidistant cuts made on stolons and/or rhizomes of a mother plant. More in detail, two successive cuts of the plurality of cuts are made at a predetermined distance from each other, for example comprised between 1 mm and 10 mm, advantageously between 2 mm and 4 mm, for example 3 mm.

In an exemplary embodiment the step of obtaining the cuttings from the mother plant comprises:
a step of detecting the, or each, node present on the stolon, or rhizome, of the mother plant by automatic detection means, for example optical;
a cutting step of said cutting at a predetermined distance from said detected node, in particular said distance corresponding to the length of said internode portion set between 1 mm and 10 mm.

In particular before the “pseudo-seeding” a step can be provided of application on at least said node of said cutting a layer of inert material selected from the group consisting of: a clay, a talc, a wax, an oil, substances based on zeolites, a biostimulating substance, a polymeric substance, a fungicide, plant hormones, an insecticide, a fertilizer, for example humic or fulvic acid, a phytoregulator, an antiperspirant, a tanning agent, or a combination thereof.

In particular, the stoloniferous and/or rhizomatous plant species is selected from the group consisting of: Cynodon dactylon, Cynodon hybrid, Zoysia japonica, Zoysia matrella, Zoysia tenuifolia, Zoysia pacifica, Paspalum vaginatum, Stenotaphrum secundatum, Buchloe dactyloides, Eremochloa ophiuroides, Paspalum notatum, Pennisetum clandestinum, Agrostis stolonifera, Poa pratensis, Poa trivialis.

In particular, before the seeding step, the cuttings can be stored in conditions selected from the group consisting of:
at an environmental temperature and humidity;
at a controlled temperature and humidity;
at a temperature below 0° C.;
at a pressure less than atmospheric pressure;
at a modified atmosphere in an airtight container, for example, increasing the content of CO₂ and decreasing the content of oxygen.

In particular, the cuttings can be tanned, i.e. added with a product that improves the preservation and extends the vigor, for example fungicides.

In particular, the cuttings can be submitted to a treatment selected from the group consisting of: encapsulation, sugar coating, seed coating, film coating.

Advantageously, the cuttings “as such” or coated with the layer, or film, of protective material, are “seeded” in a nursery, or in open field, in particular on firm ground, or worked ground, or on a substrate, or on a synthetic turfs for making a mixed natural-artificial turf.

In particular, the plurality of cuttings can be combined with a support that is adapted to be located at the desired site for making the turf. More precisely, the cuttings can be combined to the support, for example to a fabric by weaving, or integrated in it, for example during the production of the support, in particular in case of geotextiles. Alternatively, the cuttings can be combined by means of gluing, in particular, with biodegradable substances. This way, it is possible to assist the step of “seeding”. In a further exemplary embodiment the cuttings can be combined between two layers of woven or non-woven fabric, i.e. geotextile.

Advantageously, the support is made of a material selected from the group consisting of: a biodegradable natural material, a synthetic material, a combination thereof.

In particular, the biodegradable natural material is selected from the group consisting of: paper, jute, hemp, cotton, coconut fibres, agave fibres.

The synthetic material can be, instead, selected from the group consisting of: polypropylene, nylon, PVC.

The above described step of carrying out a vegetative propagation can be selected from the group consisting of: a manual seeding, or an automatic seeding.

In particular, the cutting can be subject to a step of filming through the application of a film of coating material to obtain a loose product, or pseudo-seed, with shape substantially equivalent to the cutting as such.

In particular, the carrying out a vegetative propagation can be a scattered seeding, or a row seeding, or a single seeding planting.

According to another aspect of the invention, a loose plant product to provide a turf for sporting, recreational or ornamental purposes, said loose product being obtained starting from a mother plant of a stoloniferous and/or rhizomatous plant species, said mother plants being equipped with stolons and/or rhizomes comprising a plurality of nodes, wherein two successive nodes are connected by internode portions, or internodes, where the cutting consists of at least one node, and on one internode of length set between 1 mm and 10 mm, in particular between 1 and 5 mm.

In particular, the length set between 1 mm and 10 mm of the internode can coincide with the distance between two consecutive nodes, or correspond to a portion of this distance.

In particular, the cutting obtained by the mother plant may have an overall length set between 1 mm and 10 mm, advantageously, set between 1 mm and 5 mm.

### BRIEF DESCRIPTION OF THE DRAWINGS

The invention will be now illustrated with the following description of an exemplary embodiment thereof, exemplifying but not limitative, with reference to the attached drawings in which:

FIG. 1A shows an elevational front view of a first stoloniferous plant species that can be used as a starting mother plant for carrying out the method, according to the invention, for making a turf;

FIG. 1B shows in detail a node and an internode of the mother plant of FIG. 1A;

FIG. 2A shows an elevational front view of a rhizomatous plant species that can be used alternatively to the plant species of FIG. 1A as starting mother plant for carrying out the method, according to the invention, for making a turf;

FIGS. 2B and 2C show in detail a node and an internode of the mother plant of FIG. 2A;

FIG. 3A shows an elevational front view of a stoloniferous plant species that can be used as starting mother plant for carrying out the method according to the invention, for making a turf;

FIG. 3B shows in detail a node and an internode of the mother plant of FIG. 3A;

FIG. 4A shows an elevational front view of a rhizomatous plant species that can be used as starting mother plant, alternatively to the plant species of FIG. 1A and of FIG. 2A, for carrying out the method according to the invention, for making a turf;

FIGS. 4B and 4C show in detail a node and an internode of the mother plant of FIG. 4A;

FIG. 5A shows an elevational front view of the mother plant of FIG. 1A;

FIGS. from 5B to 5D show a possible succession of operations to which the mother plant of FIG. 5A can be submitted to obtain a loose product that can be used as synthetic seed for making a turf, according to the invention;

FIG. 5E shows a possible step to which it is possible to submit the cutting before being sown alternatively to the step of FIG. 5D;

FIGS. 6A and 6B show, respectively, an elevational front view of a rhizomatous or stoloniferous plant obtained starting from a plant not treated with a growth retardant and a plant treated with a growth retardant in which the nodes are nearer for the shorter length of the internodes;

FIG. 7 shows a perspective elevational side view of a seedling tray that can be used for cultivating the plants that can be used as mother plants for the method according to the invention;

FIG. 8 shows a perspective elevational side view of a plurality of plants originated by the nodes arranged in the cells of the seedling container of FIG. 7;

FIG. 9 diagrammatically shows the plant of FIG. 5A, or of FIG. 8, submitted to a cutting step, according to the invention;

FIG. 10 shows a package of loose products obtained with the method according to the invention for making a turf;

FIG. 11A shows a cross sectional view of a particular exemplary embodiment of the step of transplantation obtained by arranging a support to which the cuttings have been previously combined;

FIGS. 11B and 11C show in detail two possible cuttings used as pseudo-seeds that can be used in the method for making a turf according to the invention

### DESCRIPTION OF A PREFERRED EXEMPLARY EMBODIMENT

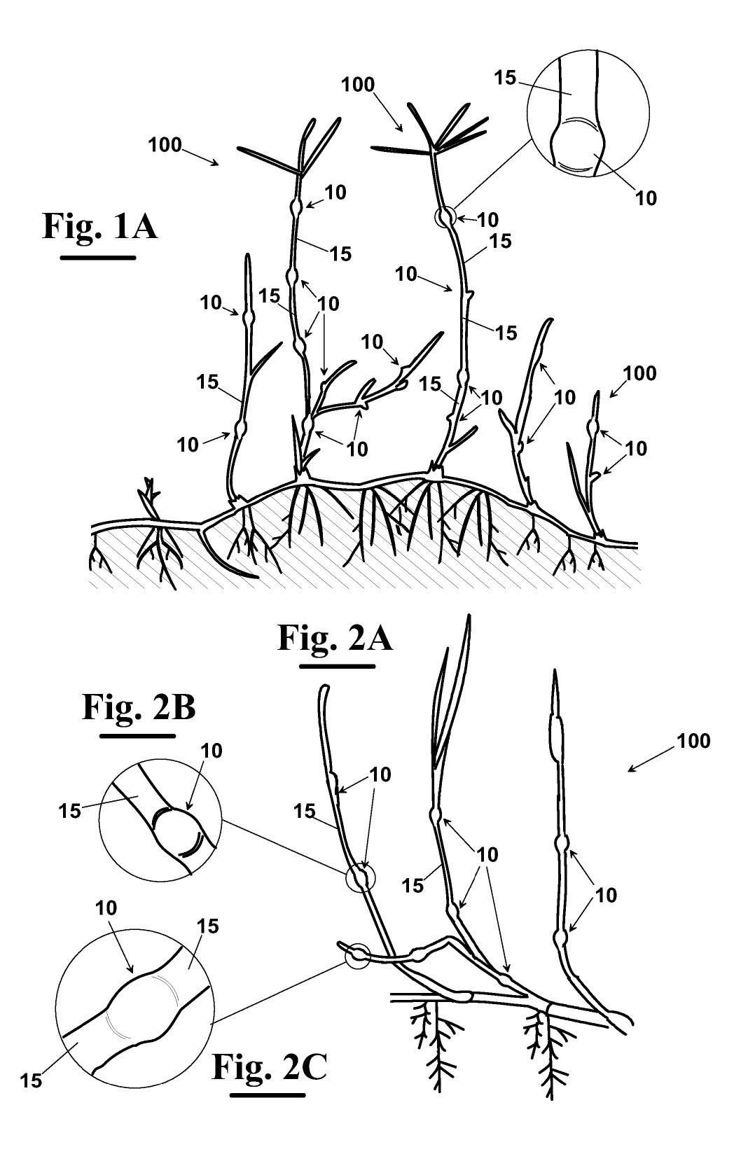

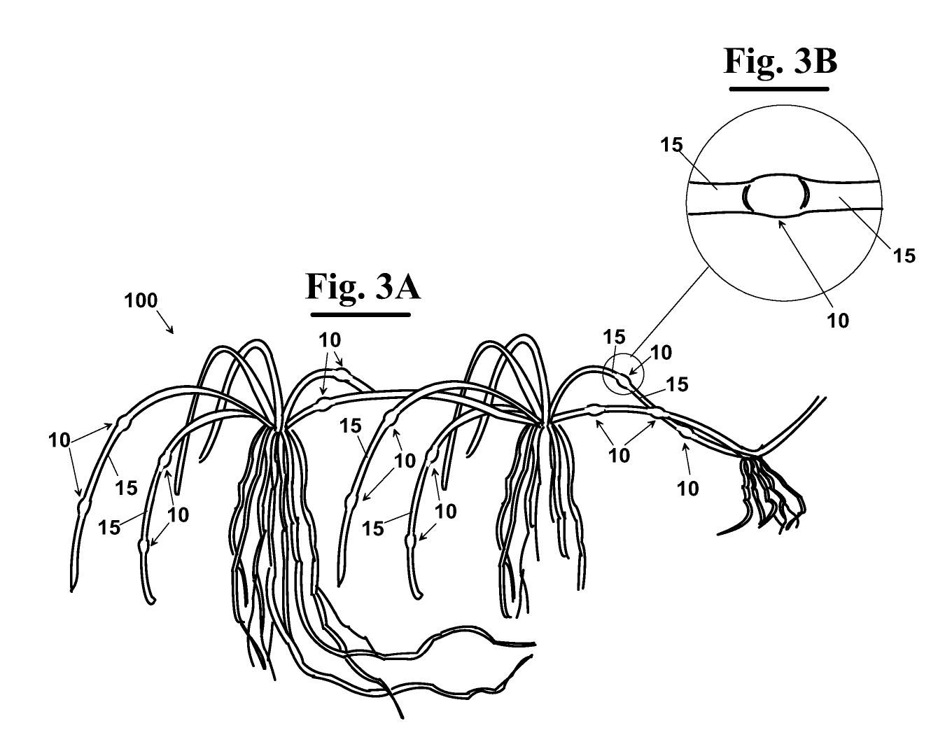

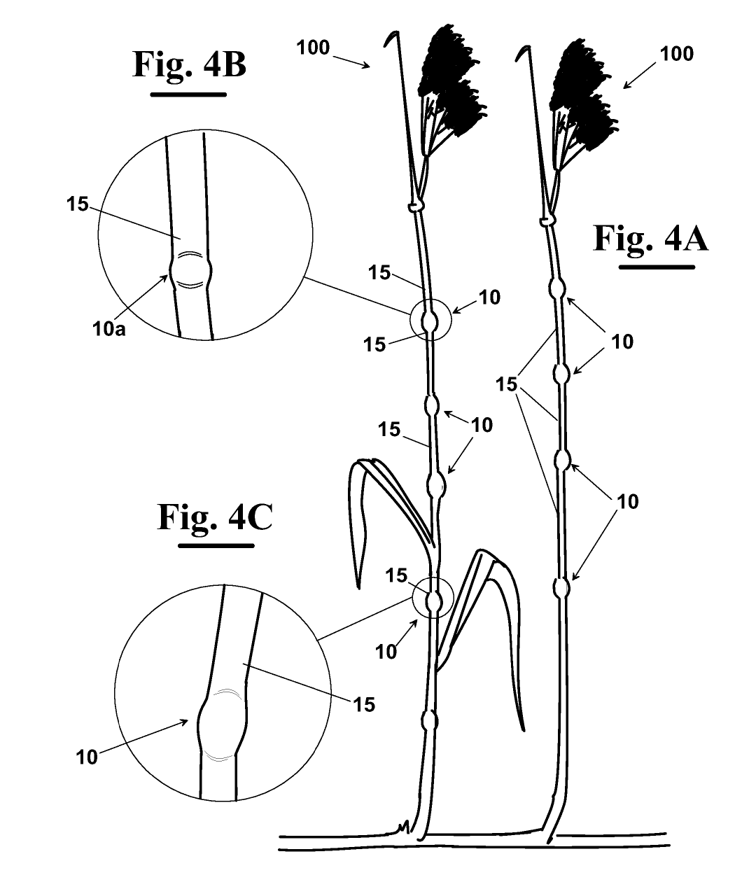

In the FIGS. 1A, 2A and 3A three different types of plants 100 are shown belonging to rhizomatous or stroloniferous plant species used as starting mother plants, for making a turf through the method according to the invention. In particular, as shown in detail in FIGS. 1B, 2B, 2C, 3B, 4B and 4C, mother plant 100 has a plurality of nodes 10 and of internodes 15, i.e. plant portions set between two successive nodes 10.

As well known, nodes 10 are points of the axis of the bud which grow into leaves, whereas internodes 15 are zones of the bud that separate nodes 10 from each other. The length of the stem is due substantially to the internodes because the cells of the nodes are short.

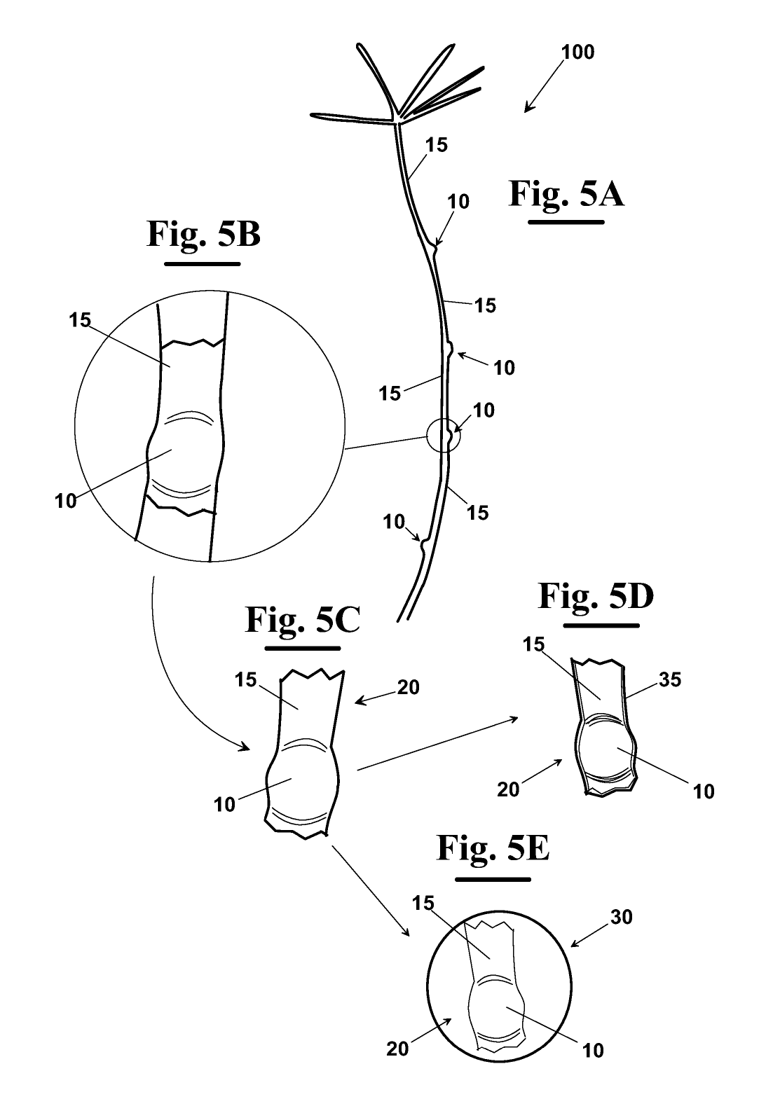

According to the invention, once achieved a predetermined vegetative growth, mother plant 100 (FIG. 5A) is cut (FIG. 5B) obtaining a plurality of portions, or cuttings 20 (FIG. 5C). Each cutting 20 consists of a node portion, or node, 10 and of an internode of length set between 1 mm and 10 mm, in particular set between 1 mm and 5 mm.

Cuttings 20 can be obtained starting from the stolons, or from the rhizomes, of mother plant 100 using simple cutting tools, such as scissors for pruning, or using automatic cutting machines, not shown in the figures, which can be equipped with a device for detecting nodes 10. Cuttings 20 can be prepared starting from stolons in the lignified state, or in the herbaceous state.

The presence of the internode 15 in cutting 20 is necessary because it contains amides and other essential nutritive substances for germination and growth of nodes 10. Cuttings 20 obtained by mother plant 100 are, then, sown by hand, or by a seeding machine in the desired site for making the turf.

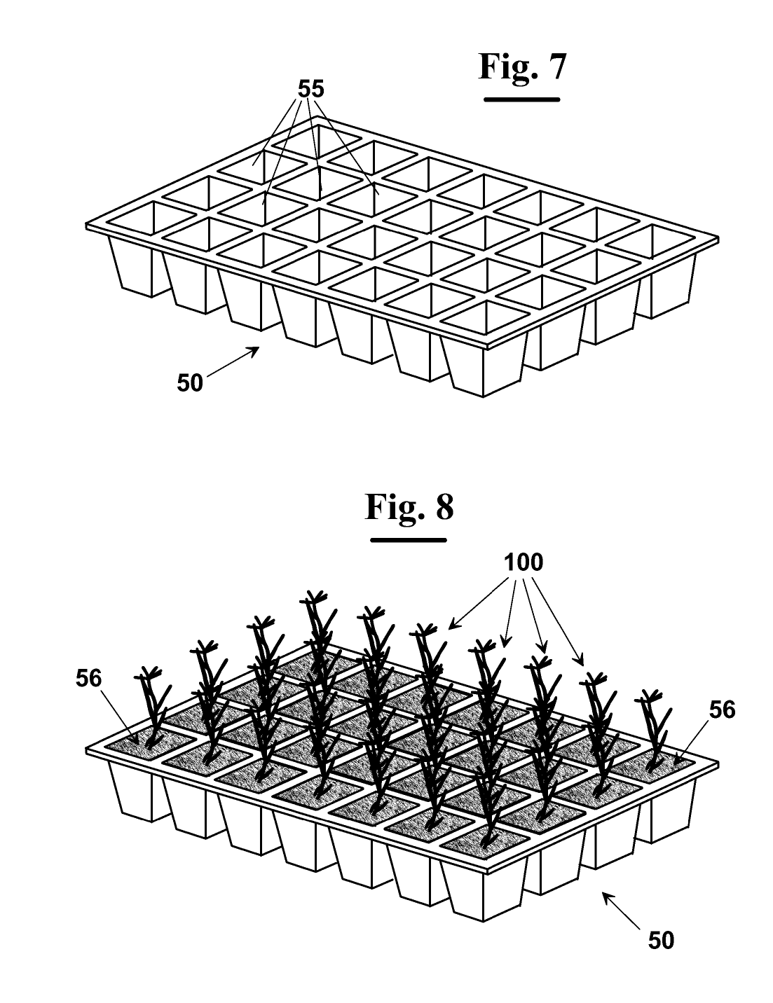

In FIGS. 7 and 8 the case is diagrammatically shown in which mother plants 100 are cultivated into a seedling container 50 comprising, as well known, a plurality of cells 55 arranged according to a predetermined array. In this case the step of cultivating comprises a step of introducing a seed, or a cutting, of the stoloniferous and/or rhizomatous selected plant species into each cell 55 and an extraction step of mother plant 100 from the cell 55 same once it has achieved a predetermined vegetative growth.

For preparing cuttings 20 and, in particular, nodes 10 having optimal dimensions, hydric contents, germination capacity, mother plant 100 can be submitted to specific treatments that provide the use of fertilizers, phytoregulators and appropriate irrigation regimes in addition to the choice of the right moment for harvesting, according to the kind of plant species chosen for making the turf.

Cuttings 20 can be submitted to a step of film coating that provides the application of a film, or layer, of coating 35 made of biodegradable, or synthetic material that adheres to cutting 20 (FIG. 5D). Alternatively, cuttings 20 can be submitted to a step of seed coating, or sugar coating (FIG. 5E). This way, a loose product 30 is obtained with a substantially spherical, or ovoidal, shape or in any case compact and with rounded edges.

In both cases as above described, coating film 35, or the “comfit” 30, allows, in particular, to protect cutting 20 from the attacks of parasites, and to simplify, furthermore, the step of seeding in particular in case of use of an automatic seeding machine.

The collected cuttings 20 are stored, in order to maintain for a long time their vigor. More in detail, cuttings 20 are kept in places with controlled temperature, pressure and humidity.

Cuttings 20 can be packaged loose, in a similar way to packaging seeds, for example in a package 60 (FIG. 10), and stored at a temperature near, or lower, to the zero for example a temperature set between −5° C. and 8° C. This allows to keep cuttings 20 in ideal conditions, for example for their transportation from the site of production to the place chosen for making the turf.

In addition, or alternatively, it is possible to adjust the pressure of the environment in which cuttings 20 are stored. In particular, cuttings 20 can be kept in an environment at a pressure less than the atmospheric pressure, i.e. under vacuum. This way, conditions of preservation are obtained such that the nodes, and then the somatic synthetic seeds, have vigor for more than one year.

In an exemplary embodiment, the mother plants can be treated with a growth regulator, in particular a growth retardant, for example selected from the group consisting of: Trinexapac-ethyl, prohexadione-calcium, paclobutrazol, Uniconazole, Miconazole, mepiquat-chloride, Chloride of coline chlorine, or chlormequat-chloride, Flurprimidol, Mefluidide, a combination thereof.

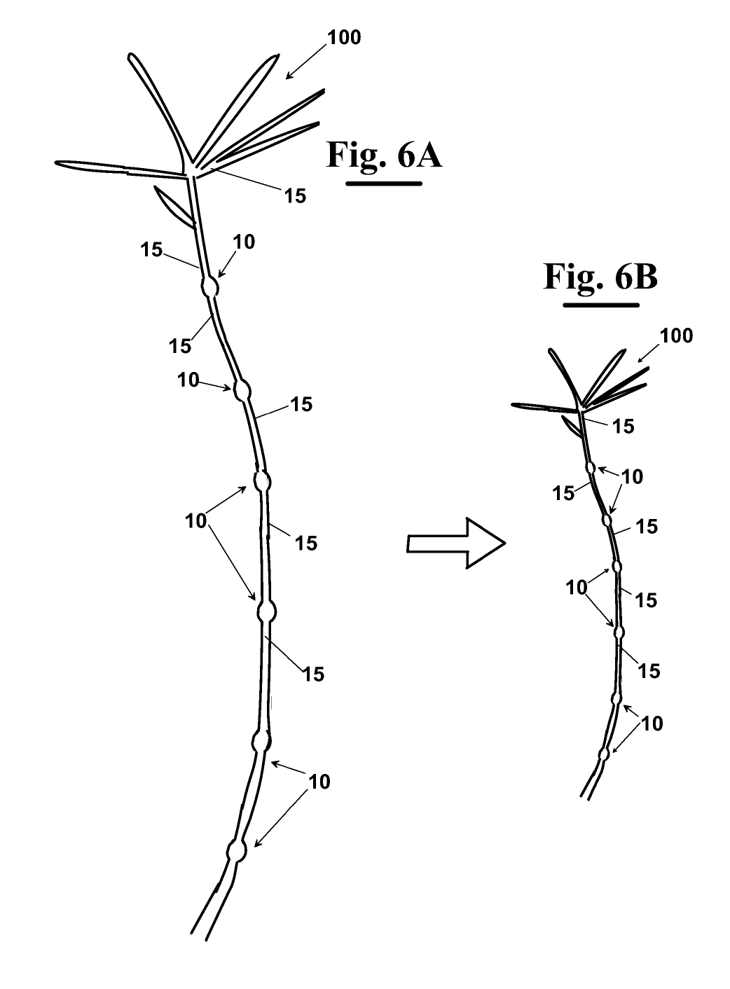

This way, a mother plant 100b is obtained whose nodes 10 are close to one another, i.e. separated from internodes 15 having a short length, and, in particular, less than the length of internodes 15 of a mother plant 100a that would be obtained not treating the plant with a growth retardant (FIGS. 6A and 6B). This allows to reduce the waste parts of the plant 100, corresponding to internodes 15 excluded from cutting 20, i.e. the one that does not fall within the length set between 1 mm and 10 mm, or between 1 mm and 5 mm.

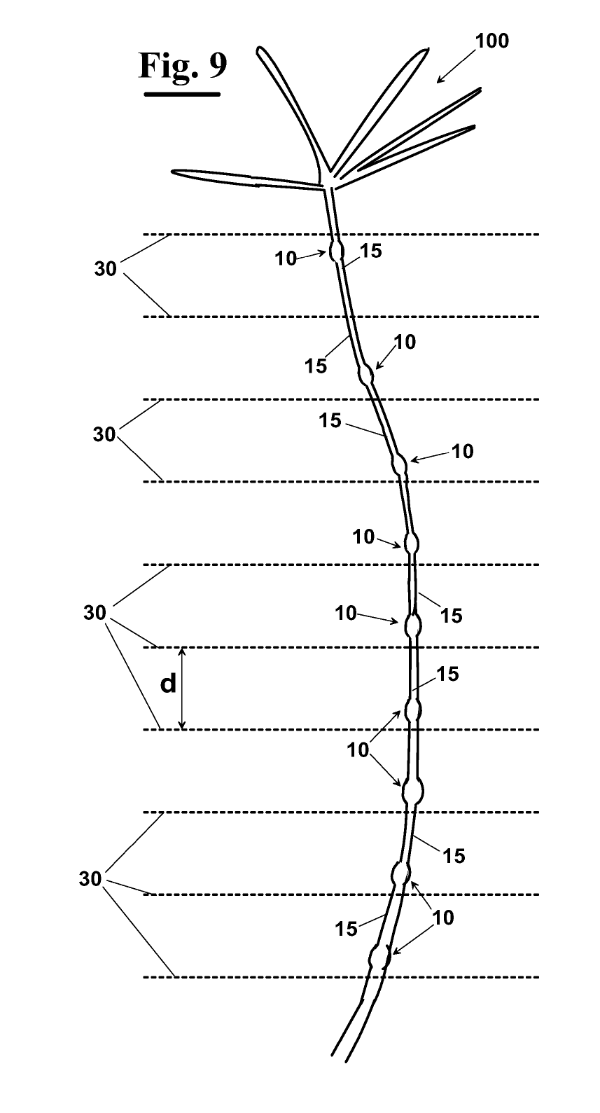

As diagrammatically shown in FIG. 9 this solution is particularly advantageous in the case in which the cutting step of mother plant 100 is carried out through a plurality of cuts 30 made at a predetermined fixed distance d. The above described plurality of cuts 30 can be carried out by a tool, not shown in the figures, for example a roll having a plurality of knives with fixed pitch, where the pitch corresponds to the distance between two successive cuts 30. In particular, the above described plurality of cuts can be made, in this case, with automatic machines.

The plant species that can be propagated by the somatic synthetic seeds, as above described, belong to the graminaceae that are able to make stolons and/or rhizomes in particular: Cynodon dactylon, Cynodon hybrid, Zoysia japonica, Zoysia matrella, Zoysia tenuifolia, Zoysia pacifica, Paspalum vaginatum, Stenotaphrum secundatum, Buchloe dactyloides, Eremochloa ophiuroides, Paspalum notatum, Pennisetum clandestinum, Agrostis stolonifera, Poa pratensis, Poa trivialis.

Cuttings 20 “as such”, or coated with a layer, or film, of protective material 30, are sown in a nursery, or in open field, in particular on firm ground, or on worked ground.

Cuttings 20 can also be seeded on a synthetic turf, in order to obtain a mixed natural-artificial turf, for example as described in WO2006/008579.

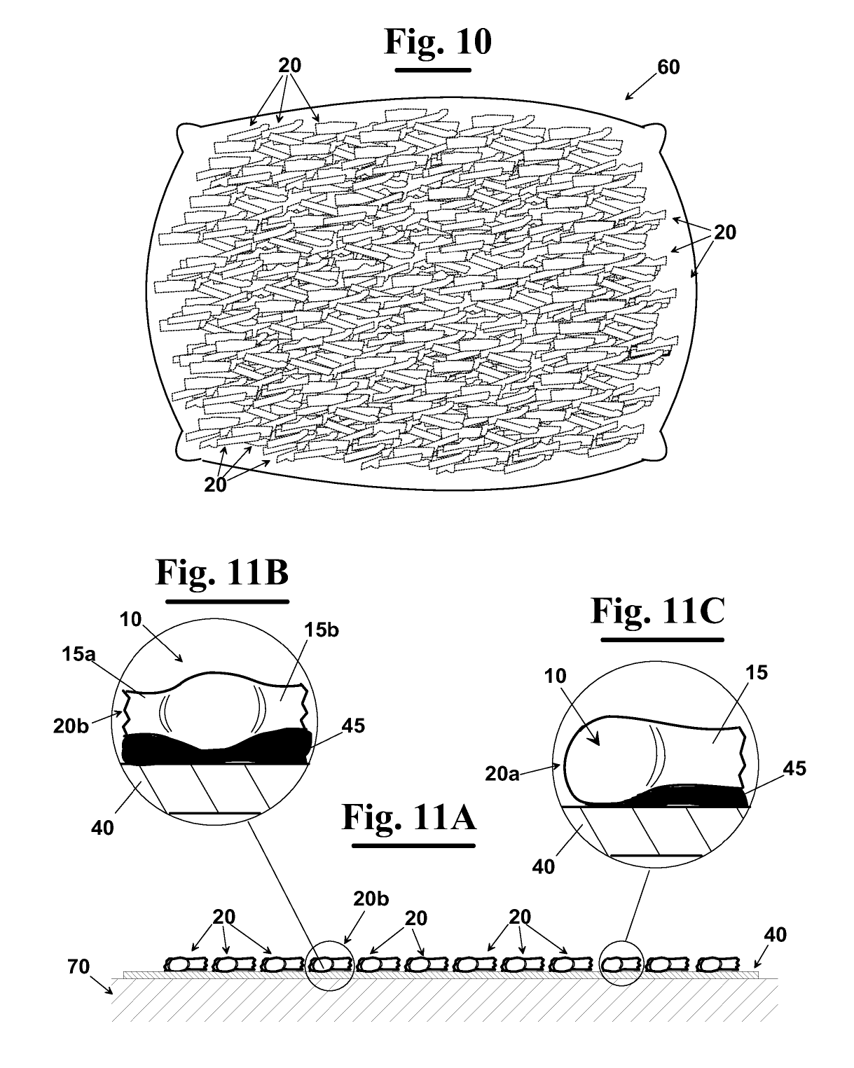

In an exemplary embodiment, cuttings 20 can be arranged on a support, for example a fabric 40 that is adapted to be located at a desired site for making the turf (FIG. 11). Support 40 can be made of a natural biodegradable material, such as paper, jute, hemp, cotton, coconut fibres, or of a synthetic material such as polypropylene, nylon, PVC, or a combination thereof.

According to the invention, from mother plant 100 a cutting 20a can be obtained having a node 10 and an internode 15 protruding from one side of the node (FIG. 11B).

In an exemplary embodiment, from mother plant 100 a cutting 20b can be obtained having a first internode 15a protruding from one side of node 10 and a second internode 15b protruding from the opposite side of node 10 (FIG. 11C).

Cutting 20, and, in particular, node 10, can be coated with at least one layer of protection material for protecting it from dehydration, from parasitic attacks, from mechanical damages, or, it can be coated with material containing substances oriented to improve the germination capacity and the rooting of the new seedling, and so it can be defined as somatic synthetic seed. The somatic seeds obtained as above described, can be produced and preserved without any protection of physical-chemical nature, or they can be submitted to sugar coating, or film coating, using material as inert dusts, clay, fungicides, insecticides, waxes, polymeric substances, antiperspirant substances.

The somatic synthetic seeds can be directly sown in the field with a scatter seeding method, row seeding, or with single seed planting, also pneumatic, or can be sown, by hand, or by a machine, in a nursery for making seedlings from transplantation in the surface that is suited to become a turf.

The seeds synthetic can be kept and used in shape uncoated or coated. The coating provides packaging cutting 20 with a layer, or film, of a biologically inert material for example: clays, talk, wax, oil, polymeric substances, antiperspirating substances and all the other substances already used to protect physically and chemically or regularizing the shape of the seeds, obtained by the plants owing to the phenomenon of fecundation.

Alternatively, the sugar coating provides the packaging of cutting 20 with a layer, or film, of a biologically active material. The biologically active substance for packaging the somatic synthetic seeds can be a fungicide, an insecticide, a fertilizer, an hormone, or a phytoregulator.

Since a node 10 that has already germinated is not adapted to be used as starting “synthetic seed” for carrying out the method according to the invention, it is preferable to draw cuttings 20 from mother plant 100 when the node has not yet germinated, i.e. for most of the hybrid grasses for which it is not possible a reproduction for seed, during the winter period.

The foregoing description of a specific embodiment will so fully reveal the invention according to the conceptual point of view, so that others, by applying current knowledge, will be able to modify and/or adapt for various applications such an embodiment without further research and without parting from the invention, and it is therefore to be understood that such adaptations and modifications will have to be considered as equivalent to the specific embodiment. The means and the materials to realise the different functions described herein could have a different nature without, for this reason, departing from the field of the invention. It is to be understood that the phraseology or terminology employed herein is for the purpose of description and not of limitation.

1. Method for making a turf for sports, ornamental or recreational use characterised in that it comprises the steps of:
selecting a stoloniferous and/or rhizomatous plant species;
cultivating a plurality of mother plants belonging to said stoloniferous and/or rhizomatous species up to reach a predetermined vegetative growth, said mother plants comprising stolons and/or rhizomes having a plurality of nodes, wherein two successive nodes are connected by internode portions, or internodes;
obtaining at least one cutting from each mother plant of said plurality of mother plants, said cutting comprising at least one node and one internode, wherein said internode has a length set between 1 mm and 10 mm;
carrying out a vegetative propagation of a plurality of said cuttings in a desired site then obtaining said turf.

2. Method, according to claim 1, wherein said plant species belongs to the family of hybrid grasses, i.e. of the grasses that are not able to reproduce by seed.

3. Method, according to claim 1, wherein said step of cultivating said mother plants is made in a greenhouse, or in an open field.

4. Method, according to claim 1, wherein said step of cultivating said mother plants is carried out in a container.

5. Method, according to claim 1, wherein said step of cultivating said mother plants is carried out in a seedling container comprising a plurality of cells, said step of cultivating comprising in this case:
a preliminary step of introducing a cutting, or pseudo-seed, of said stoloniferous and/or rhizomatous plant species in said cells of said seedling container;
an extraction step of said mother plants from said cell of said seedling container once achieved said predetermined vegetative growth.

6. Method, according to claim 1, wherein said internode has a length set between 1 mm and 5 mm.

7. Method, according to claim 1, wherein said cutting is introduced into a container for preservation and/or their transportation and/or sale.

8. Method, according to claim 1, wherein said cutting is kept at a temperature set between −5° C. and 8° C.

9. Method, according to claim 1, wherein said mother plants are treated with a plant growth regulator, in particular a growth retardant, selected from the group consisting of:
Trinexapac-ethyle;
Calcium-proesadione;
Paclobutrazol;
Uniconazole;
Miconazole;
Mepiquat Chloride;
Chlorocholine Chloride, or chlormequat;
Flurprimidol;
Mefluidide;
or a combination thereof.

10. Method, according to claim 1, wherein said step of obtaining said cutting is carried out through a cutting step of said mother plants.

11. Method, according to claim 10, wherein said cutting step comprises a plurality of equidistant cuts made on stolons and/or rhizomes of a mother plant, wherein two successive cuts of said plurality of cuts are made at a predetermined distance from each other.

12. Method, according to claim 1, wherein said step of obtaining said, or each, cutting from said mother plants provides the further steps of:
a step of detecting the, or each, node that is present on the stolon or rhizome, of the mother plants by means of automatic detection, for example optical;
a cutting step of said cutting at a predetermined distance from said detected node, in particular said distance corresponding to the length of said internode set between 1 mm and 10 mm.

13. Method, according to claim 10, wherein two successive cuts of said plurality of cuts are made at a predetermined distance comprised between 1 mm and 10 mm.

14. Method, according to claim 1, wherein a step is provided of applying on said cutting an inert material selected from the group consisting of:
a clay;
a talcum;
a wax;
an oil;
compounds based on zeolites;
a biostimulator
a polymeric substance;
a fungicide;
plant hormones;
an insecticide;
a fertilizer, for example humic or fulvic acid;
a phytoregulator;
an antiperspirant;
a tanning agent;
or a combination thereof.

15. Method, according to claim 1, wherein said stoloniferous and/or rhizomatous plant species is selected from the group consisting of: Cynodon dactylon, Cynodon hybrid, Zoysia japonica, Zoysia matrella, Zoysia tenuifolia, Zoysia pacifica, Paspalum vaginatum, Stenotaphrum secundatum, Buchloe dactyloides, Eremochloa ophiuroides, Paspalum notatum, Pennisetum clandestinum, Agrostis stolonifera, Poa pratensis, Poa trivialis.

16. Method, according to claim 1, wherein a step is provided of coating at least said node of said cutting with a protective substance.

17. Method, according to claim 1, wherein said cuttings are subject to a treatment selected from the group consisting of:
encapsulation;
sugar coating;
seed coating;
film coating.

18. Method, according to claim 1, wherein said plurality of cuttings engages with a support, in particular a fabric, or a net, said support is adapted to be arranged at the desired site for making said turf.

19. Method, according to claim 18, wherein said support is made of a material selected from the group consisting of:
a biodegradabile natural material;
a synthetic material;
a combination thereof.

20. Method, according to claim 19, wherein said biodegradable natural material is selected from the group consisting of: paper, jute, hemp, cotton, coconut fibres, agave fibres.

21. Method, according to claim 1, wherein said synthetic material is selected from the group consisting of: polypropylene, nylon, PVC.

22. Method, according to claim 1, wherein said step of carrying out a vegetative propagation is selected from the group consisting of:
seeding by hand;
seeding by a machine.

23. Method, according to claim 1, wherein said carrying out a vegetative propagation is selected from the group consisting of:
seeding on firm ground;
seeding on worked ground;
seeding on a substrate;
seeding on a synthetic turf, in order to obtain a mixed natural-artificial turf;
scatter seeding;
row seeding.

24. Loose plant product to provide a turf for sports, ornamental or recreative use, said loose product being obtained starting from a mother plant of a stoloniferous and/or rhizomatous plant species, said mother plants comprising stolons and/or rhizomes having a plurality of nodes, wherein two successive nodes are connected by plant portions, or internodes, characterised in that it comprises at least one node and an internode of length set between 1 mm and 10 mm.
